SEQUENCE LISTING (7 sequences)

SEQ ID NO: 1 - protein, 7 aa, Artificial Sequence
  Synthetic Peptide
RRETAWA

SEQ ID NO: 2 - protein, 9 aa, Artificial Sequence
  Synthetic Peptide
RYRGDLDRR

SEQ ID NO: 3 - protein, 7 aa, Artificial Sequence
  Synthetic Peptide
YRGDLGR

SEQ ID NO: 4 - protein, 5 aa, Artificial Sequence
  Synthetic Peptide
RGDGW

SEQ ID NO: 5 - protein, 6 aa, Artificial Sequence
  Synthetic Peptide
GRGDTP

SEQ ID NO: 6 - protein, 18 aa, Artificial Sequence
  Synthetic Peptide
RGDTFQTSSSPTPPGSSS

SEQ ID NO: 7 - protein, 5 aa, Artificial Sequence
  Synthetic Peptide
RGDEE

(19) United States
(12) Patent Application Publication
(10) Pub. No.: US 2010/0166808 A1
(43) Pub. Date: Jul. 1, 2010

(54) Method of Facilitating Intracellular Uptake or Transcellular Transport of Cargo Using Nanocarriers Containing Optimal Surface Densities of Integrin-Specific Ligands

(76) Inventors: Giovanni Marco Pauletti, Cincinnati, OH (US); Anil G. Menon, Cincinnati, OH (US)

Correspondence Address: DINSMORE & SHOHL LLP, 1900 CHEMED CENTER, 255 EAST FIFTH STREET, CINCINNATI, OH, 45202, US

(21) Appl. No.: 12/619,903

(22) Filed: Nov. 17, 2009

Related U.S. Application Data
(60) Provisional application No. 61/115,249, filed on Nov. 17, 2008.

(51) Int. Cl. A61K 9/00; C12N 5/071; A61K 31/711; A61K 38/17; A61K 39/00; A61K 31/715; A61K 39/395; A61K 33/42; A61K 33/08; A61K 33/06; A61K 38/14; A61K 31/01; A61K 31/08; A61K 38/20; A61K 39/106; A61K 39/08; A61K 38/42; A61K 31/7105; A61P 43/00

(52) U.S. Cl. 424/400; 435/366; 514/44 R; 514/12; 424/184.1; 514/58; 424/130.1; 424/601; 424/690; 424/682; 514/8; 514/762; 514/723; 424/85.2; 424/236.1; 424/239.1; 514/6

(57) ABSTRACT

A method of facilitating intracellular uptake or transcellular transport of cargo comprising: contacting target cells with a composition comprising an effective amount of cargo incorporated in a nanocarrier having a surface, wherein the nanocarrier surface comprises integrin-specific ligands having a surface density of approximately 5 to 50,000 integrin-specific ligands per μm², and maintaining contact between the composition and the target cells for a period of time sufficient to permit intracellular uptake or transcellular transport of the cargo.

### CROSS REFERENCE TO RELATED APPLICATIONS

This application claims the benefit of provisional patent application Ser. No. 61/115,249, filed Nov. 17, 2008.

### BACKGROUND OF THE INVENTION

1. Field of the Invention

The invention concerns a method of utilizing nanocarriers that display integrin-specific ligands at defined optimal surface densities to effectuate intracellular uptake or transcellular transport of cargo-loaded nanocarriers. Optimal surface densities efficiently augment internalization of nanocarriers into and transfer of nanocarriers across epithelial and endothelial cells, thereby improving diagnostic, preventive, and therapeutic interventions in a fetus, child, and adult.

The method of the present invention delivers nanocarriers with optimal densities of integrin-specific ligands to target cell populations. The method facilitates efficient uptake of nanocarriers into epithelial microfold cells (“M cells”) and noninvasive transfer of nanocarriers across the human trophoblast barrier, avoids the need for fetal injections, and reduces the need for parenteral administration of vaccines.

2. Background

Nanomedicine includes diagnostic and therapeutic applications that utilize artificial nanocarriers, which are miniature devices or particles that can readily interact with biomolecules on cell surfaces and within cells. Nanotechnology offers better sensitivity, specificity, and reliability than conventional technology, thereby allowing the identification of disease at earlier stages than previously possible. Diagnostic applications, including imaging, rely on nanocarriers capable of acquiring multiple measurements and integrating multiple analytical steps. The combination of highly-sensitive detection nanostructures with cargo (i.e., diagnostic, therapeutic, or preventative agents) has created new opportunities for early disease diagnosis and therapy, as well as therapy control known as “theragnostics” (Pene et al. Crit. Care Med., 37 (Suppl 1):S50-S98 (2009)). Pharmaceutical nanocarriers such as viral vectors, polymeric nanoparticles, and liposomes are advantageous for both protecting cargo from degradation and delivering cargo more selectively to target cells. Nanocarriers also effectively enhance the delivery of poorly-soluble therapeutics and control the release rate of encapsulated cargo.

Many nanocarriers are natural or synthetic polymers that have defined physical and chemical characteristics. As a consequence, a person skilled in the art can engineer desired properties, such as target selectivity, biodegradability, biocompatibility, and responsiveness to environmental factors (e.g., pH or temperature changes), into nanocarriers to improve performance. Unique chemical properties of polymeric components in nanocarriers facilitate surface modification with additional chemical entities designed to alter biodistribution. For example, selectivity for a desired target cell population can be increased by covalently or non-covalently associating such nanocarriers with cell-specific molecular recognition signatures (“ligands”) that interact with unique proteins expressed on surface of desired cell populations (“receptors”).

Many targeting ligand types have been studied (e.g., small molecules, antibodies, protein fragments, peptides, and aptamers) for their ability to physically connect a nanocarrier loaded with cargo with a desired target cell (Liu et al. Int. J. Cancer, 120:2527-2537 (2007); Pangburn et al. J. Biomed. Eng., 131:074005-1-074500-20 (2009); Hakimori et al., U.S. Pat. No. 5,230,900; Kingsman et al., U.S. Pat. No. 6,852,703). However, only a few ligands have been identified that also facilitate internalization into a target cell following binding to a cell surface receptor. Mechanistically, binding of ligands that facilitate internalization to a cell surface receptor induces endocytosis of the coupled nanocarriers, which facilitates cell-specific uptake of cargo.

Receptor-mediated endocytosis can also lead to efficient transport of the nanocarriers through the cell using transcytosis. Nanocarriers that are usually too large to pass through a cell barrier are shuttled through the cell inside bilayer vesicles and thereby gain access to distant physiological compartments. Endocytosis-mediated internalization of nanocarriers is medically advantageous because cargo is selectively concentrated in the target cell, thereby improving imaging sensitivity and therapeutic efficacy. Successful examples of this approach include folate-targeted MRI contrast agents (Choi et al. Acad. Radiol., 11:996-1004 (2004)) and carboplatin nanocapsules (Hamelers et al. Mol. Cancer. Ther., 5(8):2007-2012 (2007)). Transcytosis-mediated transport of nanocarriers across endothelial and epithelial barriers is critical in enabling the cargo to reach physiological compartments that are distant to the site of administration. Biological barriers exhibiting significant transcytosis capacities are found in the gastrointestinal, nasal, and pulmonary mucosa, where specialized epithelial cells that are concentrated in mucosa-associated lymphoid tissue, such as M cells, facilitate presentation of foreign, antigenic material to underlying immunocompetent cells. Successful targeting of transcytosis transporters can induce tolerance to foreign antigens, such as food allergens, and encourage production of protective antibodies, as is desired in vaccination applications (Powell et al. Br. J. Nutr., 98 (Suppl 1):559-563 (2007); Pascual, US2004/0033486). Transcytosis is also used by the placenta to regulate the exchange of macromolecules between the maternal and fetal circulation system. This physiological transport process protects the unborn child by delivering maternal antibodies (Mostov Annu. Rev. Immunol., 12:63-84 (1994)). Transcytosis transporters expressed in microendothelial cells forming the blood-brain barrier are the focus of intense research effort to increase the availability and effectiveness of diagnostic and therapeutic interventions related to pathophysiological conditions associated with the central nervous system. For example, experiments have demonstrated that nanocarriers functionalized with ligands that exhibit specific affinity for the transferrin receptor enter the brain via receptor-mediated transcytosis (Boado Pharm. Res., 24:1772-1787 (2007)).

Integrins constitute a large family of transmembrane adhesion receptors. They are heterodimeric and composed of α and β subunits. Integrins play a vital role in cell-cell and cell-matrix interactions, function as endocytosis and transcytosis receptors, and help to maintain physiological homeostasis. Additionally, integrins facilitate internalization and transport of foreign nanocarriers, such as bacteria and viruses, across biological barriers (Alfsen et al. Mol. Biol., 16 (4267-4279) (2005); Tyrer et al. Vaccine, 25:3204-3209 (2007)).

Integrin-targeted nanocarriers loaded with different cargo are predicted to greatly improve the effectiveness of medical diagnostic, preventive, and therapeutic interventions. Unique to integrin-mediated events is a distinct dependence upon the number of specific interactions between integrin receptors and their ligands. The overall strength of the ligand-receptor interaction (“avidity”) is governed by the intrinsic affinity of the individual ligand-receptor bond and the number of these bonds (“valency”). A common feature of integrin receptors is their propensity to cluster or aggregate when bound to ligands. This clustering seems essential to elicit the full range of cellular responses mediated by integrins, including internalization. While monomeric binding affinity of integrin-specific ligand affects initial contact with a receptor, it appears that spatial organization of ligands into discrete clusters of locally high ligand density enables more efficient integrin aggregation. This process appears to be driven predominantly by ligand density rather than monomeric binding affinity. In simple terms, at low ligand concentrations, many of the integrins are unable to find free ligands, which limits the formation of integrin clusters. Conversely, at very high ligand concentrations, integrins are able to bind individual ligands and remain randomly spread over the cell surface. Consequently, there exists a range of optimal densities of polyvalent ligand that induces most effective integrin aggregation. To date, the range of optimal densities has not been recognized or identified, and the quantitative impact of multivalent ligand interactions with integrin receptors in medical applications of nanocarriers, including diagnosis, prevention, and therapy of human disease, remains largely unexamined. In fact, a lack of correlation between surface density of nanocarrier ligands and receptors has led to unpredictable results and highly variable success rates using surface-decorated nanocarriers as drug delivery mechanisms. The present invention recognizes and defines the optimal density of integrin-specific ligands for internalization of nanocarriers with cargo.

The method of the present invention provides a novel strategy for improving delivery of nanocarrier-associated cargo into desired target cells or across biological membrane barriers in a fetus, child, or adult. Nanocarriers functionalized with an optimal density of integrin-specific ligands efficiently permit noninvasive transfer of diagnostic, preventive, and therapeutic agents across the blood-brain and human trophoblast barrier in the placenta and promote efficient uptake of such agents into epithelial M cells concentrated in gastrointestinal mucosa-associated lymphoid tissue. The methods for diagnosis, prevention, therapy, and monitoring comprise administering cargo-loaded nanocarriers that display optimal densities of integrin-specific ligands to epithelial mucosa or into systemic circulation. This novel method avoids the need for fetal injections, reduces the need for parenteral administration of vaccines or membrane-compromising permeation enhancers and avoids the unpredictable results and highly variable success rates that previously hindered the use of nanocarriers decorated with ligands for drug delivery. The novel method of this invention improves the efficacy of uptake by and transport across cells and is an important practical and economical achievement.

### SUMMARY OF THE INVENTION

Accordingly, the present invention provides a method and composition for facilitating the intracellular uptake or transcellular transport of cargo using nanocarriers containing optimal surface densities of integrin-specific ligands.

One embodiment of the invention provides a method of facilitating intracellular uptake or transcellular transport of cargo comprising contacting target cells with a composition comprising an effective amount of cargo incorporated in a nanocarrier having a surface, wherein the nanocarrier surface comprises integrin-specific ligands having a surface density of approximately 5 to 50,000 integrin-specific ligands per μm², and maintaining contact between the composition and the target cells for a period of time sufficient to permit intracellular uptake or transcellular transport of the cargo.

Another embodiment of the invention provides a method of facilitating intracellular uptake or transcellular transport of cargo comprising contacting target cells with a composition comprising an effective amount of cargo incorporated in a nanocarrier having a surface, wherein the nanocarrier surface comprises integrin-specific ligands having a surface density of at least 40,000 integrin-specific ligands per μm², and maintaining contact between the composition and the target cells for a period of time sufficient to permit intracellular uptake or transcellular transport of the cargo.

Another embodiment of the invention comprises a pharmaceutical composition comprising an effective amount of cargo incorporated in a nanocarrier having a surface, wherein the nanocarrier surface comprises integrin-specific ligands having a surface density of approximately 5 to 50,000 integrin-specific ligands per μm², and a pharmaceutically-acceptable excipient.

### BRIEF DESCRIPTION OF THE DRAWINGS

FIG. 1 is a graphic illustration of the receptor selectivity of the integrin-specific ligand RYRGDLDRR (SEQ ID NO:2) in vitro using purified human integrins.

FIG. 2 is a graphic illustration of the effect of integrin-specific ligand surface density on in vitro transport of viral nanocarriers across a model of the human placenta.

FIG. 3 is a graphic illustration of the effect of integrin-specific ligand surface density on transepithelial in vitro transport of polymeric nanocarriers across model M cells.

### DETAILED DESCRIPTION

### Definitions

The term “nanocarrier” as used herein refers to any natural or synthetic carrier of regular or irregular shape consisting of an equivalent spherical surface area between approximately 1×10⁻⁵ μm² and 15 μm², most preferably between 0.002 μm² and 3.5 μm². Nanocarriers may or may not be self-replicating (e.g., viral nanocarriers and synthetic nanocarriers, respectively). Nanocarriers may be comprised of inorganic material (e.g., Au, Ag, Co, Fe) or organic material (e.g., proteins, sugars, poly(lactide-co-glycolide), poly(methyl/methacrylate), polyethylene oxide, polypropylene oxide) or a mixture of each. Exemplary nanocarriers include, but are not limited to, adenoviruses, herpesviruses, rotaviruses, parechoviruses, hantaviruses, lentiviruses, bacterophages, virus-like particles, liposomes, micelles, nanoparticles, nanocapsules, polymersomes, dendrimers, polyplexes, nanoemulsions, nanotubes, and nanocrystals.

The term “equivalent spherical surface area” as used herein refers to a surface area of a spherical particle that is equivalent to the surface area of a nonspherical particle.

The term “integrin” as used herein refers to a heterodimer of non-covalently associated α and β subunits of an integrin receptor facilitating cellular internalization or transcytosis. The most preferred integrins for the purpose of this invention are the α₂β₁, α₃β₁, α₄β₁, α₅β₁, α_{V}β₁, α_{V}β₃, α_{V}β₅, α_{M}β₂, and α_{X}β₂ integrins.

The term “integrin-specific ligand” as used herein refers to a compound that binds to integrin receptors with an equilibrium dissociation constant of less than 10⁻³ M and that triggers receptor-mediated internalization of ligand-associated macromolecules or carriers into a mammalian cell. Chemically, integrin-specific ligands may be categorized as small molecule ligands having a molecular weight up to approximately 1000 Daltons (most preferably between 300 and 700 Daltons), peptides, peptidomimetics, non-peptidomimetics, retro-peptide analogs, inverso-peptide analogs, retroinverso-peptide analogs, pseudopeptides, depsipeptides, proteins and protein fragments having a molecular weight of greater than approximately 5000 Daltons, carbohydrates, nucleic acid molecules such as deoxyribonucleic acid (“DNA”) and ribonucleic acid (“RNA”), and nucleic acid analogs such as phosphorothioates, peptide-nucleic acids, and boranophosphates. Integrin-specific ligands may be associated with the surface of nanocarriers, either covalently or non-covalently, in the presence or absence of a “linker.” The integrin-specific ligand can be associated with the nanocarriers by any means or methods known by one of skill in the art without limitation. For example, integrin-specific ligands can be covalently attached to monomeric units comprising a polymeric nanocarrier, as described by Garinot et al. (J. Controlled Release, 120:195-204 (2007)), or to prefabricated nanocarriers, as disclosed in U.S. Pat. No. 7,507,530 and US 2009/0181101. Examples of methods for non-covalent association of integrin-specific ligands to nanocarriers are described by Schmidt (Prot. Eng. 14:769-774 (2001)), and U.S. Pat. No. 6,284,503. The skilled artisan can select the appropriate method for association of integrin-specific ligands to nanocarriers based on the physical and chemical properties of the chosen nanocarrier and ligand.

The term “small molecule ligand” as used herein refers to an organic compound having a molecular weight of less than approximately 1000 Daltons (preferably between approximately 300 and 700 Daltons) and an affinity for integrin receptors. Examples of small molecule ligands are described in Kessler et al. Lett. Pept. Sci., 2:155-160 (1995), Smallheer et al. Bioorg. Med. Chem. Lett., 14:383-387 (2007), and Heckman et al. Angew. Chem. Int. Ed., 46:3571-3574 (2007).

The term “linker” as used herein refers to a chemical entity that joins the nanocarrier to the integrin-specific ligand, either covalently or non-covalently, by van der Waals, hydrogen, or ionic forces, or a mixture thereof. The linker can be covalently attached to the nanocarrier using any suitable functional group available for reaction. These functional groups include, but are not limited to, sulfhydryl-, carboxyl-, and amine groups. Suitable methods for these reactions are described in U.S. Pat. No. 4,671,958, U.S. Pat. No. 4,659,839, U.S. Pat. No. 5,391,312, U.S. Pat. No. 5,614,503, U.S. Pat. No. 6,585,475, and U.S. Pat. No. 6,673,905. In certain embodiments, the integrin-specific ligand can be introduced as fusion protein using recombinant genetic engineering technologies. A “cleavable” linker refers to a linker that can be degraded or otherwise used to separate the integrin-specific ligand from the nanocarrier. Cleavable linkers are generally substrates for enzymatic degradation by such compounds as peptidases, proteases, nucleases, esterases, phosphatases, and lipases. Cleavable linkers may also be degraded by environmental stimuli such as changes in temperature, magnetic field, pH, and a salt concentration following internalization or external manipulation. Exemplary linkers include, but are not limited to, straight or branched-chain carbon linkers, heterocyclic carbon linkers, substituted carbon linkers, unsaturated carbon linkers, aromatic carbon linkers, peptide linkers, copolymer linkers, and block copolymer linkers.

The term “cargo” as used herein refers one or more compounds that are encapsulated within or associated with nanocarriers. Cargo includes, but is not limited to, diagnostic agents, preventative agents, therapeutic agents, and mixtures thereof.

The term “diagnostic agent” as used herein refers to a compound or complex that aids in the detection and functional assessment of biological and physiological processes suitable for diagnosis, assessment, and monitoring, but not for treatment, of human disease. Diagnostic agents exhibit unique optical, electrochemical, electrical, mass-sensitive, and thermal properties relevant to prognostic and therapeutic management guidance.

The term “preventive agent” as used herein refers to a compound that, when administered to an organism, delays the onset, limits the extent, or inhibits the incidence of pathological conditions. Preventive agents include, but are not limited, to drugs, vaccines, immunomodulators, alone or in combination with suitable pharmaceutical components acting synergistically with the preventive agent.

The term “therapeutic agent” as used herein refers to a therapeutically-active compound suitable for treatment, management, or control of a pathological condition, or a mixture thereof.

The term “mucosa-associated lymphoid tissue” as used herein refers to inductive sites for mucosal immune response, including, but not limited to, gut-associated lymphoid tissue, Peyer's patches, isolated lymphoid follicles, nasopharynx-associated lymphoid tissue, bronchus-associated lymphoid tissue, mesenteric lymph nodes, and cervical lymph nodes.

The intent of defining the terms stated above is to clarify their use in this description and does not explicitly or implicitly limit the scope of the claimed invention, which scope is defined solely by the claims.

In one embodiment of this invention, intracellular uptake or transcellular transport of cargo is facilitated by contacting target cells with a composition comprising an effective amount of cargo incorporated in a nanocarrier having a surface, wherein the surface comprises integrin-specific ligands having a density of approximately 5 to 50,000 integrin-specific ligands per μm², and maintaining contact between the composition and the target cells for a period of time sufficient to permit intracellular uptake or transcellular transport of the cargo.

Means of delivering the composition to target cells, such that the composition will contact such cells, and means of maintaining contact with the cells for a period of time are known in the art. Examples of such means include, but are not limited to, delivery by oral, nasal, pulmonary, vaginal, and mucosal means. In part, the novelty of this invention lies in the use of an optimal density of integrin-specific ligands to facilitate the transport of cargo-loaded nanocarriers into or across the target cells.

In another exemplary embodiment of the invention, the surface density of the integrin-specific ligands of the method is between approximately 15,000 and 35,000 ligands per μm².

In another exemplary embodiment of the invention, the surface density of the integrin-specific ligands of the method is between approximately 5 and 100 ligands per μm².

In another exemplary embodiment of the invention, the surface density of the integrin-specific ligands of the method is between approximately 100 and 200 ligands per μm².

In another exemplary embodiment of the invention, the surface density of the integrin-specific ligands of the method is between approximately 200 and 500 ligands per μm².

In another exemplary embodiment of the invention, the surface density of the integrin-specific ligands of the method is between approximately 500 and 1000 ligands per μm².

In another exemplary embodiment of the invention, the surface density of the integrin-specific ligands of the method is between approximately 1000 and 5000 ligands per μm².

In another exemplary embodiment of the invention, the surface density of the integrin-specific ligands of the method is between approximately 5000 and 15,000 ligands per μm².

In another exemplary embodiment of the invention, the integrin-specific ligands of the method have equilibrium dissociation constants for heterodimers of integrin receptors of less than 10⁻³ M.

In another exemplary embodiment of the invention, the integrin-specific ligands of the method are selected from the group comprising small molecule ligands having a molecular weight of up to approximately 5000 Daltons.

In another exemplary embodiment of the invention, the integrin-specific ligands of the method are selected from the group comprising small molecule ligands having a molecular weight of between approximately 100 and 500 Daltons.

In another exemplary embodiment of the invention, the integrin-specific ligands of the method are selected from the group comprising small molecule ligands having a molecular weight of between approximately 500 and 1000 Daltons.

In another exemplary embodiment of the invention, the integrin-specific ligands of the method are selected from the group comprising small molecule ligands having a molecular weight of between approximately 1000 and 2000 Daltons.

In another exemplary embodiment of the invention, the integrin-specific ligands of the method are selected from the group comprising small molecule ligands having a molecular weight of between approximately 2000 and 5000 Daltons.

In another exemplary embodiment of the invention, the integrin-specific ligands of the method are selected from the group comprising of peptides, peptidomimetics, non-peptidomimetics, retro-peptide analogs, inverso-peptide analogs, retroinverso-peptide analogs, pseudopeptides, and depsipeptides.

In another exemplary embodiment of the invention, the integrin-specific ligands of the method are selected from the group comprising proteins and protein fragments having a molecular weight of greater than approximately 5000 Daltons.

In another exemplary embodiment of the invention, the integrin-specific ligands of the method are selected from the group comprising carbohydrates.

In another exemplary embodiment of the invention, the integrin-specific ligands of the method are selected from the group comprising nucleic acid molecules and nucleic acid analogs, such as phosphorothioates, peptide-nucleic acids, and boranophosphates.

In another exemplary embodiment of the invention, integrin-specific ligands of the method are selected from the group comprising monoclonal antibody mAbl6, polypeptide having sequence RRETAWA (SEQ ID NO:1), polypeptide having sequence RYRGDLDRR (SEQ ID NO:2), and polypeptide having sequence YRGDLGR (SEQ ID NO:3), polypeptide having sequence RGDGW (SEQ ID NO:4), polypeptide having sequence GRGDTP (SEQ ID NO:5), polypeptide having sequence RGDTFQTSSSPTPPGSSS (SEQ ID NO:6), and polypeptide having sequence RGDEE (SEQ ID NO:7).

In another exemplary embodiment of the invention, the nanocarrier of the method has an equivalent spherical surface area of between approximately 1×10⁻⁵ μm² and 15 μm².

In another exemplary embodiment of the invention, the nanocarrier of the method has an equivalent spherical surface area of between approximately 1×10⁻⁵ μm² and 3×10⁻⁵ μm².

In another exemplary embodiment of the invention, the nanocarrier of the method has an equivalent spherical surface area of between approximately 3×10⁻⁵ μm² and 5×10⁻³ μm².

In another exemplary embodiment of the invention, the nanocarrier of the method has an equivalent spherical surface area of between approximately 5×10⁻³ μm² and 0.13 μm².

In another exemplary embodiment of the invention, the nanocarrier of the method has an equivalent spherical surface area of between approximately 0.13 μm² and 2 μm².

In another exemplary embodiment of the invention, the nanocarrier of the method has an equivalent spherical surface area of between approximately 2 μm² and 5 μm².

In another exemplary embodiment of the invention, the nanocarrier of the method has an equivalent spherical surface area of between approximately 5 μm² and 15 μm².

In another exemplary embodiment of the invention, the nanocarrier of the method is a viral vector selected from the group comprising adenoviruses, herpesviruses, rotaviruses, parechoviruses, hantaviruses, lentiviruses, and bacteriophages carrying linear or circular nucleic acid molecules.

In another exemplary embodiment of the invention, the nanocarrier of the method is a non-viral vector selected from the group comprising virus-like particles, liposomes, micelles, nanoparticles, nanocapsules, polymersomes, dendrimers, polyplexes, nanoemulsions, nanotubes, and nanocrystals.

In another exemplary embodiment of the invention, the ligands of the method are specific to integrins selected from the group comprising heterodimers of integrin receptors consisting of an α₁₋₉, α_{M}, α_{V}, or α_{X} subunit and a β₁, β₂, β₃, or β₄ subunit.

In another exemplary embodiment of the invention, the heterodimers of integrins of the method are selected from the group comprising α₃β₄, α₄β₁, α₅β₁, α_{V}β₃, α_{V}β₅, α_{M}β₂, and α_{X}β₂ heterodimers.

In another exemplary embodiment of the invention, the method is administered to a fetus, child, or adult.

In another exemplary embodiment of the invention, the composition of the method is formulated in a dosage unit form, wherein a dosage unit comprises between approximately 0.001 and 1000 mg of cargo, and preferably between approximately 0.1 and 100 mg of cargo.

In another exemplary embodiment of the invention, the composition of the method is formulated in a dosage unit form, wherein a dosage unit comprises between approximately 0.001 and 0.01 mg of cargo.

In another exemplary embodiment of the invention, the composition of the method is formulated in a dosage unit form, wherein a dosage unit comprises between approximately 0.01 and 0.1 mg of cargo.

In another exemplary embodiment of the invention, the composition of the method is formulated in a dosage unit form, wherein a dosage unit comprises between approximately 0.1 and 1 mg of cargo.

In another exemplary embodiment of the invention, the composition of the method is formulated in a dosage unit form, wherein a dosage unit comprises between approximately 1 and 10 mg of cargo.

In another exemplary embodiment of the invention, the composition of the method is formulated in a dosage unit form, wherein a dosage unit comprises between approximately 10 and 100 mg of cargo.

In another exemplary embodiment of the invention, the composition of the method is formulated in a dosage unit form, wherein a dosage unit comprises between approximately 100 and 1000 mg of cargo.

In another exemplary embodiment of the invention, the cargo of the method is a diagnostic agent selected from the group consisting of optical biosensors, electrochemical biosensors, electrical biosensors, mass-sensitive biosensors, thermal biosensors, probes for magnetic resonance imaging, probes for positron emission tomography, probes for computed tomography, and probes for optical fluorescence imaging.

In another exemplary embodiment of the invention, the cargo of the method is a preventative agent selected from the group consisting of antigenic purified microbial components, antigenic polysaccharide-carrier protein conjugates, antigenic proteins, antigenic peptides, antigenic toxoids, antigenic DNA, antigenic RNA, sulfated cyclodextrin, activity-dependent neurotrophic factor I, activity-dependent neurotrophic factor III, and CD20 antibodies.

In another exemplary embodiment of the invention, the cargo of the method is a therapeutic agent selected from the group consisting of antiangiogenic agents, chemotherapeutic agents, antiinflammatory agents, antibacterial agents, antifungal agents, antiviral agents, human growth factors, immunostimulatory agents, therapeutic antibodies, DNA, RNA, agents having substrate affinity for cytochrome P-450 enzymes, and membrane efflux systems.

In another exemplary embodiment of the invention, the method further comprises the administration of an immunoenhancing adjuvant selected from the group comprising aluminum phosphate, aluminum hydroxide, potassium aluminum sulfate, mineral oil, muramyl tripeptide, squalene, polyoxyethylene sorbitan monooleate, polyethylene sorbitan trioleate, QS-21, monophosphoryl lipid A, macrophage-activating protein 2, CpG, IL-1, IL-2, IL-6, IL-12, cholera toxin, heat-labile enterotoxin, tetanus toxin, and Keyhole Limpet Hemocyanin.

In another exemplary embodiment of the invention, the target cells of the method are cells located in the human placenta, blood-brain barrier, or mucosa-associated lymphoid tissue.

In another exemplary embodiment of the invention, the method further comprises the step of diagnosing, monitoring, or preventing disease, or therapeutically intervening in a disease.

Another embodiment of the invention provides a method of facilitating intracellular uptake or transcellular transport of cargo comprising contacting target cells with a composition comprising an effective amount of cargo incorporated in a nanocarrier having a surface, wherein the nanocarrier surface comprises integrin-specific ligands having a surface density of at least 40,000 integrin-specific ligands per μm², and maintaining contact between the composition and the target cells for a period of time sufficient to permit intracellular uptake or transcellular transport of the cargo.

Another embodiment of the invention comprises a pharmaceutical composition comprising an effective amount of cargo incorporated in a nanocarrier having a surface, wherein the nanocarrier surface comprises integrin-specific ligands having a surface density of approximately 5 to 50,000 integrin-specific ligands per μm², and a pharmaceutically-acceptable excipient.

The following examples will serve to further typify the nature of this invention but should not be construed as a limitation of the scope thereof, which scope is defined solely by the claims.

### Example 1

### Transport of Viral Nanocarriers Displaying Integrin-Specific RYRGDLGRR Peptide at Different Surface Densities Across a Cell Culture Model of the Human Trophoblast Barrier

Preparation of Viral Nanocarrier

DNA coding sequences for the α₅β₁ integrin-specific peptide -RYRGDLGRR- are cloned as fusions to the C-terminus of T7 bacteriophage capsid proteins 10B using T7Select phage display vectors (T7Select System, Novagen). Sequences are coded at three valencies: low (0.1-1 peptide/phage; “RY-1”); medium (5-10 peptides/phage; ˜400 ligands per μm²; “RY-10”); and high (˜415 peptides/phage; ˜16,300 ligands per μm²; “RY-415”). A vector expressing a random nonapeptide (“Control-415”) at high valency (˜415 peptides/phage) is prepared as a control.

Recombinant phage clones are amplified by infection of mid-log phase E. coli (BL21 bacterial strain) culture (250 mL, OD-600, ˜0.6). Phages are then separated from cell debris by centrifugation (10 mins at 1000×g, 4° C.), 10% w/v PEG 6,000 solution is added to the supernatant, and the lysate/PEG mixture is stored overnight at 4° C.

The next day, phages are pelleted (100×g, 4° C.) and resuspended in 5 mL of 1 M NaCl, 10 mM Tris-HcL, pH 8.0, and 1 nM EDTA (ethylenediaminetetraacetic acid). Phage clones are purified by gradient centrifugation using 20-62% (w/v) CsCl gradient (Beckman Optima, LE-80 Kg 10,000×g, 20° C.). Phages are collected, dialyzed overnight against PBS (phosphate buffered saline), pH 7.4, and stored at 4° C. until further use.

Selectivity for Human Integrin Receptors

Nunc MaxiSorp™ 96-well plates are coated with 200 μL of purified human α₃β₁, α₅β₁, and αᵥβ₅ integrins (Chemicon, Temecula, Calif.) at 1 μg/mL in Tween 20™/PBS buffer, pH 7.5 (4° C.). After blocking for two hours with 5% (w/v) BSA (bovine serum albumin) in PBS, pH 7.5, phage samples are incubated with integrins in PBS, pH 7.5, supplemented with 2.5% (w/v) non-fat dry milk. After one hour of incubation, unbound phages are removed and the wells are washed five times with Ca²⁺- and Mg²⁺-containing PBS, pH 7.5.

Bound phages are quantified spectrophotometrically at λ=450 nm using monoclonal mouse T7 tail fiber antibody (1:5,000) followed by goat anti-mouse IgG horseradish peroxidase:conjugate (1:5,000) and TMB (3,3′,5,5′-tetramethyl-benzidine) as substrate.

Transepithelial Transport by Viral Nanocarrier

Human placental BeWo cells (ATCC, Rockville, Md., passages 32-40) are maintained in DMEM (Dulbecco/Vogt Modified Eagle's minimal essential medium) supplemented with 10% heat-inactivated fetal bovine serum, 1% L-glutamine, 100 I.U./mL of penicillin, 100 μg/ML of streptomycin, and 1% sodium bicarbonate at 37° C. in a controlled atmosphere of 5% CO₂ and 90% humidity. BeWo cells are seeded at a density of 1×10⁵ cells/cm² on collagen-coated Transwells™ (polycarbonate membranes, 12 mm diameter, 0.4 μm pore size). Five days after seeding, confluent cell monolayers are washed with pre-warmed Hank's Balanced Salt Solution (Sigma-Aldrich), pH 7.4.

An aliquot of an individual phage clone of particular density (1×10¹⁰ pfu) is added to the apical donor compartment of the BeWo cell layers. After one hour of incubation at 37° C., 10 μL is collected from the receiver compartment on the basolateral side and the number of phages that were transported from the apical side to the basolateral side was determined by infection of E. coli (BL21 bacterial stain) culture and plating on lysogeny broth (“LB”) agar medium. The process is repeated with the phage clones of other densities.

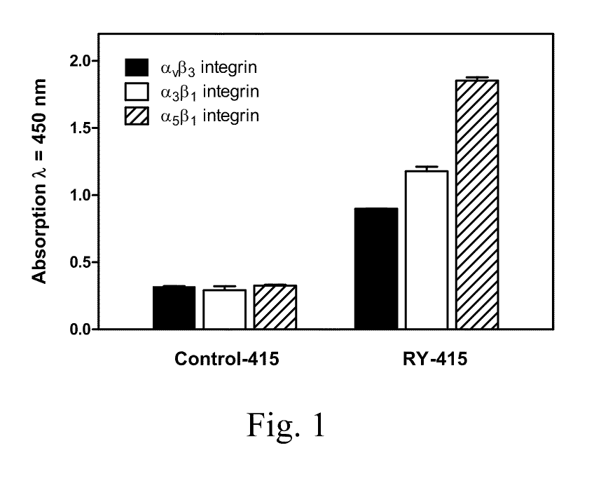

As shown in FIG. 1, the integrin-specific ligand peptide RYRGDLGRR binds most effectively to surface-immobilized human α₅β₁ integrin receptor but also displays affinity for αᵥβ₃ and α₃β₁ integrin receptor, respectively.

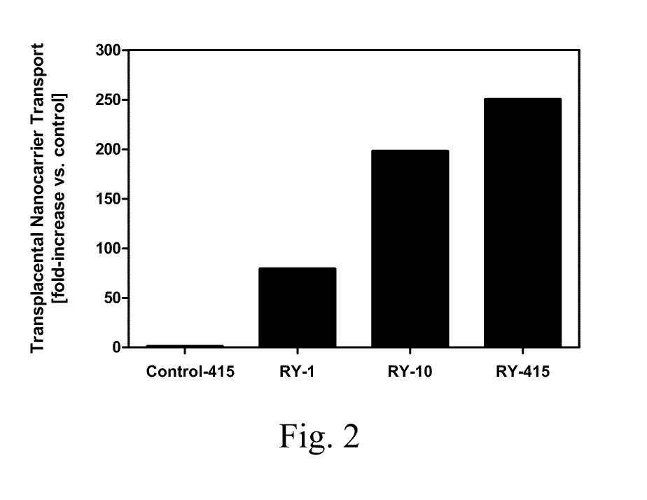

Results of transplacental transport of viral nanocarriers across an in vitro cell culture model of the human placenta is depicted in FIG. 2. The small bar associated with the control nanocarrier (“Control-415”), displaying a random peptide devoid of significant binding affinity for integrin receptors at a surface density of approximately 16,300 ligands per μm², clearly demonstrates ineffective transplacental transfer of the control. In contrast, viral nanocarriers displaying the integrin-specific RYRGDLGRR peptide at the same surface density as the control (“RY-415”) exhibit a 250-fold increase in transplacental transport over control. Lesser surface densities of this integrin-specific ligand result in less transcytosis of this viral nanocarrier across the model human placenta. For example, viral nanocarriers displaying the integrin-specific peptide at a surface density of ˜400 ligands per μm² (“RY-10”) exhibit approximately a 200-fold increase in transplacental transport over control. It is apparent from the results that increasing the surface density beyond approximately 50,000 ligands per μm² would not result in increased transcytosis.

### Example 2

### Transport of Polymeric Nanocarriers Displaying Integrin-Specific RYRGDLGRR Peptide at Different Surface Densities Across an Inverted In Vitro Model of the Human, Gastrointestinal Follicle-Associated Epithelium Containing Microfold Cells (M Cells)

Preparation of Polymeric Nanocarriers

Primary hydroxyl ends of the nonionic polyoxyethylene-polyoxypropylene block copolymer surfactant Pluronic F108 (BASF Corp., Mount Olive, N.J.) are modified with a thiol-specific pyridyl disulfide group following protocols published by Li and co-workers (Li et al. Bioconjugate Chem. 7:592-599 (1996)). 10 mg of fluorescent polystyrene particles (average diameter-200 nm; Polysciences Inc., Warrington, Pa.) are combined for 24 hours in purified water with 1 mL of a 0.4% (w/v) solution of pyridyl disulfide-activated Pluronic F108 (or 1:10 and 1:100 mixtures with unmodified Pluronic F108). Particles are recovered by centrifugation and activated for peptide coupling by 30 min incubation with 1 mL of 25 mM dithiothreitol. RYRGDLGRR peptide is synthesized by solid phase Fmoc chemistry. Peptides are activated at the N-terminus using a 5-fold molar excess of the heterobifunctional linker N-succinimidyl 3-(pyirdylditho)propionate in phosphate buffer, pH 7.0. Coupling of activated peptides to activated particles is performed at room temperature for 24 hrs in phosphate buffer, pH 7.0 using 3-fold molar excess of peptide. After washing in buffer three times, amino acid analysis is performed to estimate peptide amounts coupled to polystyrene particle surface.

Nanocarrier Transport Across M Cell Culture Model

Caco-2 and Raji cells (ATCC, Rockville, Md.) are routinely maintained in DMEM media supplemented with 10% heat-inactivated fetal bovine serum, 1% L-glutamine, 100 I.U./mL penicillin, 100 μg/mL of streptomycin, and 1% sodium bicarbonate at 37° C. in a controlled atmosphere of 5% CO₂ at 90% humidity. Caco-2 cells are seeded at a density of 1×10⁵ cells/cm² on collagen-coated Transwells (polycarbonate membranes, 12 mm diameter, 3 μm pore size). After 3-5 days, inserts are inverted. After 10 days, Raji cells are added to the basolateral compartment and Caco-2 cell monolayers are co-cultured for five days. On the day of the experiment, confluent cell monolayers are washed with prewarmed HBSS and 500 μL of the peptide-conjugated, fluorescent nanocarrier suspension is added to the apical donor compartment. After a 90 min incubation at 37° C., 200 μL are collected from the basolateral receiver compartment and 50 μL are collected from the apical donor compartment and processed for quantitative spectrophotometric analysis. Control experiments are performed using peptide-free fluorescent polystyrene particles. Preincubation of cell monolayers with 10 μg/mL of a selective, monoclonal α₅β₁ integrin antibody (mAb) is used to probe for contribution of integrin-mediated transcytosis. Results are expressed as apparent permeability coefficients (Pₐₚₚ).

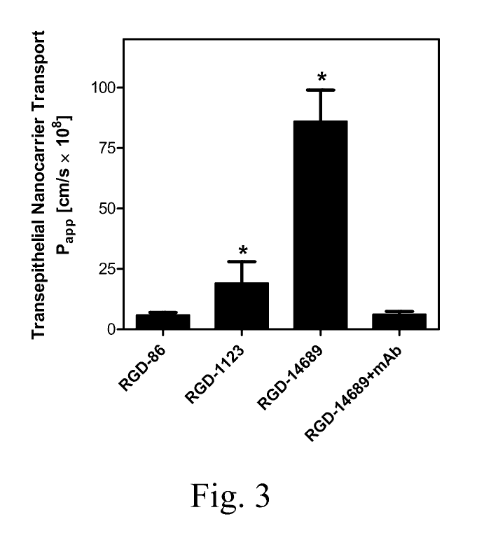

Transport data of fabricated nanocarriers with an estimated mean surface density of 86, 1123, and 14689 integrin-specific RYRGDLGRR peptides per μm² (“RGD-86,” “RGD-1123,” and “RGD-14689,” respectively) are shown in FIG. 3, which expresses the results as permeability coefficients. Increasing the surface density of the integrin-specific peptide (“RGD”) improved transport efficiency of the nanocarrier across the in vitro model of gastrointestinal M cells. Preincubation of cell monolayers with the α₅β₁ integrin antibody dramatically decreased transepithelial transport of RGD-14689, which supports the hypothesis of integrin-mediated transcytosis.

All documents cited in this application are, in relevant part, incorporated herein by reference; the citation of any document is not to be construed as an admission that such document is prior art with respect to the present invention.

While the invention has been described with reference to certain embodiments, it is understood by those skilled in the art that various changes may be made and equivalents may be substituted without departing from the scope of the invention, as that scope is defined by the claims. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

We claim:

1. A method of facilitating intracellular uptake or transcellular transport of cargo comprising:
contacting target cells with a composition comprising an effective amount of cargo incorporated in a nanocarrier having a surface, wherein the nanocarrier surface comprises integrin-specific ligands having a surface density of approximately 5 to 50,000 integrin-specific ligands per μm², and
maintaining contact between the composition and the target cells for a period of time sufficient to permit intracellular uptake or transcellular transport of the cargo.

2. The method of claim 1, wherein the ligand surface density is between approximately 15,000 and 35,000 ligands per μm².

3. The method of claim 1, wherein the integrin-specific ligands have equilibrium dissociation constants for heterodimers of integrin receptors of less than approximately 10⁻³ M.

4. The method of claim 1, wherein the integrin-specific ligands are selected from the group comprising small molecule ligands having a molecular weight of up to approximately 1000 Daltons, peptides, peptidomimetics, non-peptidomimetics, retro-peptide analogs, inverso-peptide analogs, retroinverso-peptide analogs, pseudopeptides, depsipeptides, proteins and protein fragments having a molecular weight of greater than approximately 5000 Daltons, carbohydrates, and nucleic acid molecules and analogs.

5. The method of claim 1, wherein the integrin-specific ligands are selected from the group comprising monoclonal antibody mAbl6, polypeptide having sequence RRETAWA (SEQ ID NO:1), polypeptide having sequence RYRGDLDRR (SEQ ID NO:2), and polypeptide having sequence YRGDLGR (SEQ ID NO:3), polypeptide having sequence RGDGW (SEQ ID NO:4), polypeptide having sequence GRGDTP (SEQ ID NO:5), polypeptide having sequence RGDTFQTSSSPTPPGSSS (SEQ ID NO:6), and polypeptide having sequence RGDEE (SEQ ID NO:7).

6. The method of claim 1, wherein the nanocarrier has an equivalent spherical surface area of between approximately 1×10⁻⁵ μm² and 15 μm².

7. The method of claim 1, wherein the nanocarrier is selected from the group consisting of viral and non-viral vectors.

8. The method of claim 7, wherein the viral vector is selected from the group comprising adenoviruses, herpesviruses, rotaviruses, parechoviruses, hantaviruses, lentiviruses, and bacteriophages carrying linear or circular nucleic acid molecules.

9. The method of claim 7, wherein the non-viral vector is selected from the group comprising virus-like particles, liposomes, micelles, nanoparticles, nanocapsules, polymersomes, dendrimers, polyplexes, nanoemulsions, nanotubes, and nanocrystals.

10. The method of claim 1, wherein the ligands are specific to integrins selected from the group comprising heterodimers of integrin receptors consisting of an α₁₋₉, α_{M}, α_{V}, or α_{X} subunit and a β₁, β₂, β₃, or β₄ subunit.

11. The method of claim 10, wherein the integrins are selected from the group comprising α₃β₄, α₄β₁, α₅β₁, α_{V}β₁, α_{V}β₃, α_{V}β₅, α_{M}β₂, and α_{X}β₂ heterodimers.

12. The method of claim 1, wherein the composition is administered to a fetus, child, or adult.

13. The method of claim 1, whereby the composition is formulated in a dosage unit form, wherein a dosage unit comprises between approximately 0.001 to 1000 mg of cargo, and preferably between approximately 0.1 to 100 mg of cargo.

14. The method of claim 1, wherein the cargo incorporated in the nanocarrier is a diagnostic agent selected from the group comprising optical biosensors, electrochemical biosensors, electrical biosensors, mass-sensitive biosensors, thermal biosensors, probes for magnetic resonance imaging, probes for positron emission tomography, probes for computed tomography, and probes for optical fluorescence imaging.

15. The method of claim 1, wherein the cargo incorporated in the nanocarrier is a preventative agent selected from the group comprising antigenic purified microbial components, antigenic polysaccharide-carrier protein conjugates, antigenic proteins, antigenic peptides, antigenic toxoids, antigenic DNA, antigenic RNA, sulfated cyclodextrin, activity-dependent neurotrophic factor I, activity-dependent neurotrophic factor III, and CD20 antibodies.

16. The method of claim 1, wherein the cargo incorporated in the nanocarrier is a therapeutic agent selected from the group comprising antiangiogenic agents, chemotherapeutic agents, antiinflammatory agents, antibacterial agents, antifungal agents, antiviral agents, human growth factors, immunostimulatory agents, therapeutic antibodies, DNA, RNA, agents having substrate affinity for cytochrome P-450 enzymes, and membrane efflux systems.

17. The method of claim 1, further comprising the administration of an immunoenhancing adjuvant selected from the group comprising aluminum phosphate, aluminum hydroxide, potassium aluminum sulfate, mineral oil, muramyl tripeptide, squalene, polyoxyethylene sorbitan monooleate, polyethylene sorbitan trioleate, QS-21, monophosphoryl lipid A, macrophage-activating protein 2, CpG, IL-1, IL-2, IL-6, IL-12, cholera toxin, heat-labile enterotoxin, tetanus toxin, and Keyhole Limpet Hemocyanin.

18. The method of claim 1, wherein the target cells are cells located in the human placenta, blood-brain barrier, or mucosa-associated lymphoid tissue.

19. The method of claim 12, further comprising a step of diagnosing, monitoring, preventing, or therapeutically intervening in a disease affecting the fetus, child, or adult.

20. A method of facilitating intracellular uptake or transcellular transport of cargo comprising:
contacting target cells with a composition comprising an effective amount of cargo incorporated in a nanocarrier having a surface, wherein the nanocarrier surface comprises integrin-specific ligands having a surface density of at least 40,000 integrin-specific ligands per μm², and
maintaining contact between the composition and the target cells for a period of time sufficient to permit intracellular uptake or transcellular transport of the cargo.

21. A pharmaceutical composition comprising an effective amount of cargo incorporated in a nanocarrier having a surface, wherein the nanocarrier surface comprises integrin-specific ligands having a surface density of approximately 5 to 50,000 integrin-specific ligands per μm², and a pharmaceutically-acceptable excipient.
